(19) United States
(12) Patent Application Publication
(10) Pub. No.: US 2011/0195106 A1
(43) Pub. Date: Aug. 11, 2011

(54) Decorin and Gliosis and Related System and Method

(76) Inventor: Richard Jay McMurtrey, Charlottsville, VA (US)

(21) Appl. No.: 12/701,603

(22) Filed: Feb. 8, 2010

(51) Int. Cl. A61F 2/00

(52) U.S. Cl. 424/423

(57) ABSTRACT

Brain stimulators are used to treat a variety of disorders, and their range of uses continues expand. However, one problem with long-term stimulation of neural tissue is the need to increase the stimulation parameters to continue to maintain the same clinical effect. This is thought to be due to local tissue reaction to the implanted foreign body. Because the implanted stimulator functions by means of contact with functional cells within the tissue, prevention of tissue reaction to the stimulator would make a significant improvement to the device's performance and longevity.

It is proposed that coating a neural stimulator device with decorin, and/or homologous molecules of functional or structural likeness to decorin, can function to decrease gliosis and other local tissue reaction in neural tissue. The present invention provides a novel system and method of device design and utilization that can prevent and suppress known associated tissue reactions associated with neural modulation of tissue with an implanted device, thereby improving the device's performance and longevity.

### BACKGROUND OF THE INVENTION

Stimulators of neural tissue are used to treat a variety of disorders, and they can include any deep brain stimulator, cortical stimulator, spinal cord stimulator, or nerve stimulator. Deep brain stimulators in particular are used to treat a large range of diseases and conditions, including dystonias, tremors, and other movement disorders such as Parkinson's disease (Weaver et al. 2009). Their use, however, is expanding to a wider variety of pathologies, such as epilepsy, multiple sclerosis, depression, obsessive-compulsive disorder, anxiety, obesity, eating disorders, neuroprosthesis implantation and control, chronic pain, minimally conscious states, cerebral palsy, stroke, amyotrophic lateral sclerosis, tourette syndrome, or spinal cord injury and its sequelae (including paraplegia, tetraplegia, spasticity, autonomic dysreflexia, and autonomic dysfunction of bowel and bladder). Their range of uses continues to be researched and developed.

One problem with long-term neural stimulation is the need to increase the stimulation parameters to continue to maintain the same clinical effect. Such parameters may include voltage, amperage, frequency, or pulse width. These parameters need to be adjusted over time occurs regardless of diagnosis or target site (Moss et al., 2004; Krack et al., 2003; Sydow et al., 2003; Wishart et al., 2003; Yianni et al.; 2003; Lozano 2001). This is thought to be due primarily to gliosis, reactive astrocytosis, and other local tissue reaction, such as microglial activation, leukocyte invasion, siderophages, tissue vacuolization, and multinucleated giant cell reaction (Moss et al. 2004; Nielsen et al. 2007; Sun et al. 2008). In fact, giant cell reaction was found to be invariably present as early as three months after electrode insertion (Moss, et al. 2004)

Due to these cellular and molecular changes, impedance and current distribution through the tissue are altered, thus attenuating the effectiveness of the stimulator. Because the implanted stimulator functions by means of contact with functional cells within the tissue, prevention of tissue reaction to the stimulator would make a significant improvement to the device's performance and longevity.

### DETAILED DESCRIPTION OF THE INVENTION

The present invention provides a novel system and method of device design and utilization that can prevent and suppress known associated tissue reactions associated with neural modulation of tissue with an implanted device, and which may also treat pathology related to gliosis, inflammatory neural tissue reaction, reactive astrocytosis, microglial activation, leukocyte invasion, siderophages, tissue vacuolization, multinucleated giant cell reaction, or other related diseases and processes. It is proposed that coating the implanted neural stimulator device with decorin, and/or homologous molecules of functional or structural similarity to any portion of decorin (hereafter referred to as a “decorin-like molecule”) can function to decrease gliosis and other tissue reaction in neural tissue, thereby significantly improving the device's performance and longevity.

The molecular structure of decorin has been detailed in the literature (Krusius and Ruoslahti 1986; Vesentini et al., 2003; NCBI online database gene ID 1634). Decorin is a proteoglycan that has an average molecular weight of 87-140 kilodaltons (kD) and belongs to the family of small leucine-rich proteoglycans. Decorin has a core protein component which may be bound to a glycosaminoglycan chain, and it may have many alternative splice variants. Functional equivalents of decorin include decorin native proteins, decorin core protein, decorin alternative splice variants, biglycan, fibromodulin, lumican, and other modifications, to or alternative homologous amino acid sequences of decorin.

Evidence suggests that the mechanism of decorin in neural tissues is via inhibition of the TGF-beta signaling pathway (Yamaguchi et al., 1990; Johns et al., 1992; Rabchevsky et al., 1998; Logan et al., 1999; Asher et al., 2000; Dobbertin et al., 2003; Logan and Baird, U.S. Pat. No. 5,958,411, 1995). Evidence also suggests that decorin inhibits the EGFR tyrosine kinase (Santra et al., 2002), and there is preliminary evidence to suggest that other signaling pathways are involved as well. Other proposed mechanisms include inhibition of complement activation (Krumdiek et al., U.S. Pat. No. 5,650,389, 1993), but this has not been shown to play a primary role in the central nervous system or in neural tissue reaction to foreign bodies. The detailed mechanisms remain to be fully elucidated.

Decorin has been shown to have several other cellular effects, including the suppression of neurocan, brevican, phosphacan, and NG2 expression, as well as reduction of astrogliosis and basal lamina formation after local traumatic injury (Davies et al., 2004). Davies, et al., also showed that decorin suppressed astrogliosis and macrophage/microglia accumulation at lesion sites in the central nervous system. It is also known that decorin naturally binds to collagen type I fibrils (Vesentini et al., 2003).

The novel aspect of the device is an external layer or coating with a decorin-like protein, which may be integrated or manufactured by several means. This includes chemical coupling of the molecule to the device's surface, such as by either the amino groups or carboxyl groups in the amino acid sequence, or by any molecular component of the proteoglycan chain. This may be done using the chemical reagents well known in the art: for example, amine-reactive crosslinkers, such as dithiobis succinimidyl propionate (DSP), which uses disulfide linkages to attach to a surface and links proteins by their primary amine groups, or other compounds, such as 1-Ethyl-3-(3-dimethylaminopropyl)-carbodiimide (EDC), etc. Alternatively, the decorin-like protein may be bound by polymers that cross-link or otherwise allow for adherence or attachment to the device. Alternatively, the decorin-like protein may be bound by means of its glycosaminoglycan component, its peptide backbone, its R-groups, or other moieties, or it may be modified with certain amino acid sequences that allow for binding to the surface of the device. In addition, the device surface itself may be manufactured with an absorbent or adherent coating that either contains or adheres to the decorin-like protein. This adherent coating may be made of any type of material, such as plastics, polymers, glues, ceramics, metals, silicates, carbon-based compounds, or other similar materials. The surface may also be covered with a second coating to slow the diffusion of the decorin-like molecule into surrounding tissue.

With reference to the implantable electrical stimulation device used in concert with the decorin-like molecule, it may be of any design that incorporates a conductive surface that contacts the tissue, whether made of metal (e.g., platinum-iridium, cobalt-chrome, or other alloys) or other conductive material (e.g., conductive polymers or ceramics) (Geddes and Roeder, 2003; Gimsa et al., 2005). The device may also have an insulative material to shield from conduction of current at non-targeted tissue sites. Both the conductive surfaces and insulative surfaces may integrate the decorin-like molecular surface. The device may utilize monopolar, bipolar, or multipolar stimulation, and may have any number of electrical leads, and may incorporate electrical feedback systems. The device may be internally or externally powered, and may be temporarily or permanently placed in the tissue. The device may be of any length, width, and curvature. The device may also be used for extraction or sampling of tissue or fluids, and may also be used for delivery of pharmaceutical agents or solutions, for example, through a cannula system.

Many variations of the device may be constructed which are bound within the scope of the invention.

### BRIEF DESCRIPTION OF THE DRAWINGS

The advantages and features of the invention are further described in the following drawings.

FIG. 1A is an example of a brain stimulator. This drawing shows only one of many possible examples of neural stimulation devices.

FIG. 1B is an expanded view of the surface of the device that illustrates multiple examples of possible embodiments of the invention, wherein the decorin-like molecule is attached or adhered to the surface of the stimulator device.

### DESCRIPTION OF THE PREFERRED EMBODIMENTS

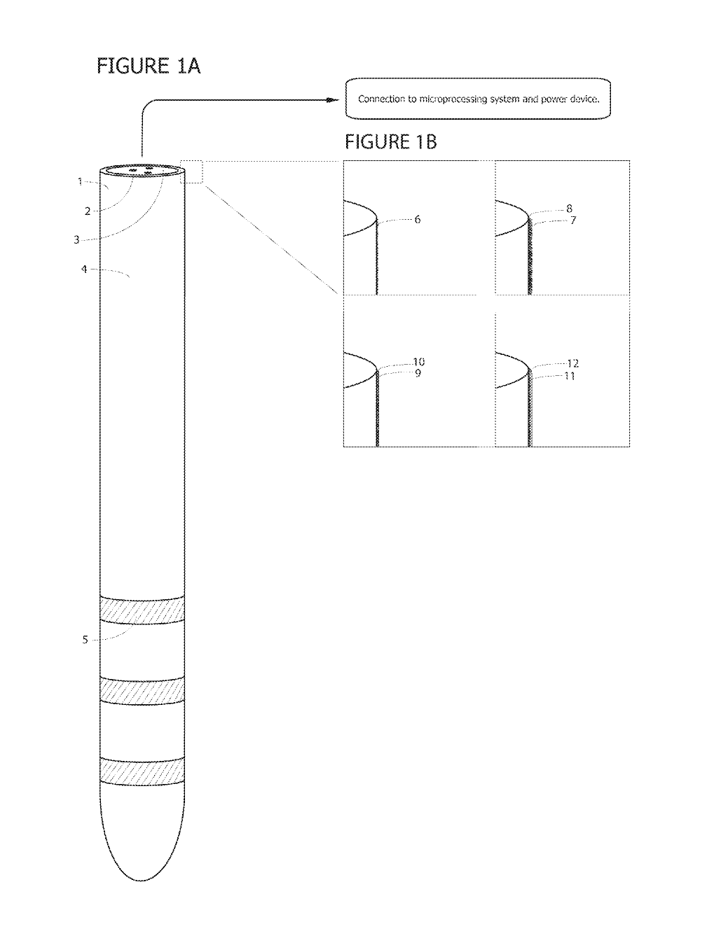

FIG. 1A is one of many possible examples of a neural stimulator device. The following is a reference for the numbered labels. 1) A neural stimulator device; in this case, a type of deep brain stimulator. 2) The conductive connection between the electrodes and a microprocessor system. 3) The substantive core of the device, which may be solid or hollow. 4) The insulative outer surface of the device. 5) The conductive surface electrode, which contacts the targeted tissue for electrical modulation.

FIG. 1B is an enlarged view of the surface of the device, wherein four possible examples of embodiments of the invention are illustrated. 6) The decorin-like molecule, coupled directly to the surface of the device. It should be noted that the decorin-like coating is only illustrated on one edge of the device, but that the entire device may be coated with the molecule. 7) The decorin-like molecule, coupled to the surface of the device by means of 8) an intermediary molecular structure. 9) The decorin-like molecule, coupled to the surface of the device by means of 10) an adhesive or adsorbant layer, which may be composed of plastics, polymers, glues, ceramics, metals, silicates, carbon-based compounds, or any other similar materials that can adhere the molecule to the device's surface. 11) The decorin-like molecule, coupled to the surface of the device with 12) a separate outer coating consisting of any plastics, polymers, glues, ceramics, metals, silicates, carbon-based compounds, or other similar materials, which may slow the diffusion of the decorin-like molecule into surrounding tissue.

### REFERENCES TO RELATED APPLICATIONS

McMurtrey, Richard J. “Decorin and Gliosis and Related System and Method.” U.S. Provisional Patent No. 61/151,334. Filed Feb. 10, 2009.
Krumdiek R, Hook M, Volanakis J. University of Alabama at Birmingham Research Foundation. “Methods for the Inhibition of Complement Activation.” U.S. Pat. No. 5,650,389. Filed Mar. 1, 1993.
Logan A, Baird A. The Whittier Institute for Diabetes and Endocrinology. “Methods of Inhibiting ECM Accumulation in the CNS by Inhibition of TGF-beta.” U.S. Pat. No. 5,958,411. Filed Mar. 24, 1995

### REFERENCED PUBLICATIONS

Asher R A, Morgenstern D A, Moon L D, Fawcett J W. “Chondroitin Sulphate Proteoglycans: Inhibitory Components of the Glial Scar.” Prog. Brain Res. 132:611-619, 2001.
Davies J E, Tang X, Denning J, Archibald S J, Davies S. “Decorin Suppresses Neurocan, Brevican, Phosphacan and NG2 Expression and Promotes Axon Growth across Adult Rat Spinal Cord Injuries” Eur J of Neuroscience 19:1226-1242, 2004.
Dobbertin A, Rhodes K E, Garwood J, Properzi F, Heck N, Rogers J H, Fawcett J W, Faissner A. “Regulation of RPTPbeta/phosphacan Expression and Glycosaminoglycan Epitopes in Injured Brain and Cytokine-treated Glia.” Mol. Cell. Neurosci. 24:951-971, 2003.
Geddes L A, Roeder R. “Criteria for the Selection of Materials for Implanted Electrodes.” Ann. Biomed. Eng., 31(7):879-890, 2003.
Gimsa J, Habel B. Schreiber U, Van Rienen U, Strauss U, Gimsa U. “Choosing Electrodes for Deep Brain Stimulation Experiments—Electrochemical Considerations.”J Neurosci Methods, 142(2):251-265, 2005.
Johns L D, Babcock G, Green D, Freedman M, Sriram S, Ransohoff R M. “Transforming Growth Factor-beta 1 Differentially Regulates Proliferation and MHC Class-II Antigen Expression in Forebrain and Brainstem Astrocyte Primary Cultures.” Brain Res. 585:229-236, 1992.
Krack P, Batir A, Van Blercom N, Chabardes S, Fraix V, Ardouin C, Koudsie A, Limousin P D, Benazzouz A, LeBas J F, Benabid A L, Pollak P. “Five-year Follow-up of Bilateral Stimulation of the Subthalamic Nucleus in Advanced Parkinson's Disease.” N Engl J Med 13; 349(20):1925-34, 2003.
Krusius T, Ruoslahti E. “Primary Structure of an Extracellular Matrix Proteoglycan Core Protein Deduced from Cloned cDNA.” Proc Natl Acad Sci USA 83(20):7683-7, 1986.
Logan A, Baird A, and Berry M. “Decorin Attenuates Gliotic Scar Formation in the Rat Cerebral Hemisphere.” Exp. Neurol. 159:504-510, 1999.
Lozano A. “Deep Brain Stimulation: Challenges to Integrating Stimulation Technology with Human Neurobiology, Neuroplasticity and Neural Repair.” International Functional Electrical Stimulation Society (IFESS) 6^{th} Annual Conference, Cleveland 2001.
Moss J, Ryder T, Aziz T Z, Graeber M B, Bain P G. “Electron Microscopy of Tissue Adherent to Explanted Electrodes in Dystonia and Parkinson's Disease.” Brain, 127(Pt 12):2755-2763, 2004.
NCBI Online Database: Decorin Sequence and Structure, Gene ID 1634, Official Symbol DCN. http://www.ncbi.nlm.nih.gov/sites/entrez?Db=gene&Cmd=ShowDetailView&TermToSearch=1634
Nielsen M S, Bjarkam C R, Sørensen J C, Bojsen-Møller M, Sunde N A, Ostergaard K. “Chronic Subthalamic High-frequency Deep Brain Stimulation in Parkinson's Disease—a Histopathological Study.” Eur J Neurol. February; 14(2): 132-8, 2007.
Rabchevsky A G, Weinitz J M, Coulpier M, Fages C, Tinel M, Junier M P. “A Role for Transforming Growth Factor Alpha as an Inducer of Astrogliosis.” J. Neurosci., 18:10541-10552, 1998.
Santra M, Reed C C, Iozzo RV. “Decorin Binds to a Narrow Region of the Epidermal Growth Factor (EGF) Receptor, Partially Overlapping but Distinct from the EGF-binding Epitope.” J. Biol. Chem. 277:35671-35681, 2002.
Sun D A, Yu H, Spooner J, Tatsas A D, Davis T, Abel T W, Kao C, Konrad P E. “Postmortem Analysis Following 71 Months of Deep Brain Stimulation of the Subthalamic Nucleus for Parkinson Disease.” J Neurosurg. August; 109(2):325-9, 2008.
Sydow O, Thobois S, Alesch F, Speelman J D. “Multicentre European Study of Thalamic Stimulation in Essential Tremor: a Six Year Follow Up.” J Neurol Neurosurg Psychiatry, 74(10):1387-91, 2003.
Vesentini S, Redaelli A, Montevecchi F. “A Molecular Analysis of Interaction Energies of the Decorin Proteoglycan—Collagen Complex in Tendon Fibrils.” Summer Bioengineering Conference, 0713-0714, Key Biscayne, Fla., Jun. 25-29, 2003.
Weaver F M, Follett K, Stern M. “Bilateral Deep Brain Stimulation versus Best Medical Therapy for Patients with Advanced Parkinson Disease: A Randomized Controlled Trial.” JAMA, 301(1):63-73, 2009.
Wishart H A, Roberts D W, Roth R M, McDonald B C, Coffey D J, Mamourian A C, Hartley C, Flashman L A, Fadul C E, Saykin A J. “Chronic Deep Brain Stimulation for the Treatment of Tremor in Multiple Sclerosis: Review and Case Reports.” J Neurol Neurosurg Psychiatry, 74(10):1392-7, 2003.
Yamaguchi Y, Mann D M, Ruoslathi E. “Negative Regulation of Transforming Growth Factor-beta by the Proteoglycan Decorin.” Nature, 346:281-284, 1990.
Yianni J, Bain P G, Gregory R P, Nandi D, Joint C, Scott R B, Stein J F, Aziz T Z. “Post-operative Progress of Dystonia Patients Following Globus Pallidus Internus Deep Brain Stimulation.” Eur J Neurol. 10(3):239-47, 2003.
   Disclosure: The invention herein involved no federally sponsored research.

I claim:

1. A system of design of deep brain stimulators, nerve stimulators, and neural implantation devices which suppress, prevent, or treat known associated tissue reaction (such as gliosis, reactive astrocytosis, microglial activation, leukocyte invasion, siderophages, and multinucleated giant cell reaction) by means of a molecular coating or surface agent.

2. The method of claim 1, wherein the molecular surface or coating is decorin or any portion of the decorin molecule, including decorin core protein, any of decorin's functional domains, or any subset of its amino acid sequences, which may be referenced under the NCBI database as gene ID 1634. (Decorin is also known as bone proteoglycan II, decorin proteoglycan, proteoglycan core protein, small leucine-rich protein 1B, or dermatan sulphate proteoglycans II, and also may be referred to by abbreviation, as in DCN, CSCD, PG40, PGII, PGS2, DSPG2, or SLRR1B.

3. The method of claim 1, wherein the molecular surface or coating is an amino acid sequence homologous to the decorin protein or which retains its functional abilities.

4. The method of claim 1, wherein the molecular surface or coating contains biglycan, fibromodulin, lumican, or homologous proteins.

5. The method of claim 1, wherein the molecular surface or coating contains any glycosaminoglycan component, including chondroitin sulfate, dermatan sulfate, or keratan sulfate.

6. The method of claim 1, wherein the device incorporates a conductive surface that contacts the tissue, whether made of metal or other conductive material such as an alloy, composite, or polymer, for the purpose of neural electrical modulation. The device may also incorporate an insulative material to shield the tissue from conduction of current at non-targeted sites. Both the conductive and insulative surfaces may incorporate decorin or homologous molecules.

7. The method of claim 1, wherein the device may have any number of electrical leads or conduction points in any pattern, distribution, or layout. The device may utilize monopolar, bipolar, or multipolar stimulation, and the electrical stimulation may be of any waveform, voltage, amperage, pulse width, and frequency. The device may utilize electrical feedback systems. The device may be internally or externally powered, and may be temporarily or permanently placed.

8. The method of claim 1, wherein the device may be of any length, width, diameter, or curvature. The device may be hollow or solid. The device may also be used for delivery of pharmaceutical agents or solutions, or for extraction or sampling of tissue or fluids, for example, through a cannula or shunt system.

9. The method of claim 1, wherein the device is any implant or graft for the purpose of electrical modulation, stimulation, or inhibition of any central nervous system tissue, whether cortical or deep brain tissue or spinal cord tissue, or any other neural tissue, such as peripheral nerve, cranial nerve, splanchnic nerve, or any motor, sensory, or autonomic nerve.

10. The method of claim 1, wherein the decorin-like molecule is annealed or coupled directly to the electrode surface and/or to the insulative surface through means well known in the art, such as chemical coupling by appropriate reagents (e.g., DSP, EDC, or other reagents, as discussed above).

11. The method of claim 1, wherein the decorin-like molecule is annealed or coupled to the electrode surface and/or to the insulative surface by means of an intermediate molecule, such as another protein or any other molecular structure.

12. The method of claim 1, wherein the decorin-like molecule is annealed or coupled to the device through the use of an adhesive or adsorbant layer. This may include any material which may adhere the decorin-like molecule to the device, such as plastics, polymers, glues, ceramics, metals, silicates, carbon-based compounds, or other similar materials. For example, interacting polymers that may crosslink with themselves or with decorin to allow for attachment and adherence to the device may be utilized.

13. The method of claim 1, wherein the decorin-like surface molecule may be covered in a separate outer coating consisting of any plastics, polymers, glues, ceramics, metals, silicates, carbon-based compounds, or other similar materials, which may slow the diffusion of the decorin-like molecule into surrounding tissue.

14. The method of claim 1, wherein the device is manufactured with the decorin-like molecule by means of a coating, such as a polymer, applied by means well known in the art, such as spray coating or dip coating, and with one or more layers, which may or may not all contain the decorin-like molecule. The coating also may cover the entire device or only portions of the devices.

15. The method of claim 1, wherein such molecule or agent is manufactured or obtained through synthetic techniques, recombinant production, isolation or purification from natural sources, or any other means.
